(19) United States
(12) Patent Application Publication
(10) Pub. No.: US 2009/0185955 A1
(43) Pub. Date: Jul. 23, 2009

(54) MICROFLUIDIC DEVICE FOR MOLECULAR DIAGNOSTIC APPLICATIONS

(75) Inventor: Antonius Johannes Maria Nellissen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/278,992

(22) PCT Filed: Feb. 8, 2007
(86) PCT No.: PCT/IB07/50416

§ 371 Date: Aug. 11, 2008

(30) Foreign Application Priority Data
Feb. 13, 2006 (EP) 06101575.6

(51) Int. Cl. G01N 33/00

(52) U.S. Cl. 422/68.1

(57) ABSTRACT

The present invention relates to a micro fluidic device for analysis of a fluid sample, especially for molecular diagnostics applications, comprising: —a substrate having a surface with at least one micro channel structure thereon; —at least one detecting, controlling and/or processing element; —at least one reception chamber for receiving the fluid sample, wherein the reception chamber is formable between a membrane and the substrate, wherein the reception chamber is fluently connected with at least one micro channel; —at least one membrane, wherein the membrane covers the upper surface of at least one micro channel structure arranged on said substrate leakage proof, whereby movement of said membrane causes a pump action on fluid located in said reception chamber in said micro channel and/or causes a valve action on fluid directed through said micro channel; and—at least one device for actuating the movement of the membrane, comprising pressure and/or vacuum generating means.

### BACKGROUND OF THE INVENTION

This invention relates to a microfluidic device for molecular diagnostic applications such as labs-on-a-chip or micro total analyses systems, to a disposable cartridge comprising said microfluidic device and to the use thereof. The microfluidic device according to the present invention is preferably used in molecular diagnostics.

The biotechnology sector has directed substantial effort toward developing miniaturized microfluidic devices, often termed labs-on-a-chip (LOC) or micro total analyses systems (microTAS), for sample manipulation and analysis. These systems are used for detection and analyses of specific bio-molecules, such as DNA and proteins.

In general micro-system devices contain fluidic, electrical and mechanical functions, comprising pumps, valves, mixers, heaters, and sensors such as optical—, magnetic—and/or electrical sensors. A typical molecular diagnostics assay includes process steps such as cell lyses, washing, amplification by PCR, and/or detection.

Integrated microfluidic devices need to combine a number of functions, like filtering, mixing, fluid actuation, valving, heating, cooling, and optical, electrical or magnetic detection, on a single template. Following a modular concept the different functions can be realized on separate functional substrates, like silicon or glass. The functions need to be assembled with a microfluidic channel system, which is typically made of plastic. With small channel geometries this way of integration becomes a very challenging process. The interfaces between the substrates and the channel plate need to be very smooth and accurate, and the channel geometries need to be reproducible, while the functional substrates should have a minimum footprint for cost efficiency. Especially with functions, which need a fluidic as well as an electric interface, the separation of the wet interface is critical. Bonding techniques must be compatible with the biochemical reagents and surface treatments present on the functional substrates.

US-A1 2003/0057391 discloses a low power integrated pumping and valving array which provide a revolutionary approach for performing pumping and valving operations in micro fabricated fluidic systems for applications such as medical diagnostic microchips. This approach integrates a lower power, high-pressure source with a polymer, ceramic, or metal plug enclosed within a micro channel, analogous to a micro syringe. When the pressure source is activated, the polymer plug slides within the micro channel, pumping the fluid on the opposite side of the plug without allowing fluid to leak around the plug. The plugs also can serve as micro valves.

However, the pump system of US-A1 2003/0057391 does not provide a sufficient small dead volume and does not provide an optimized fast fluid transport. Further, the plugs must have a positive fitting to avoid sample fluid leakage thus the low power integrated pumping and valving arrays can not be provided at low vertical range of manufacture.

In the last decade, considerable research efforts have been made to the development of microfluidic system devices in order to integrate more functions but at the same time reducing the analyze samples volumes of liquid.

Despite this effort, there is still a need for microfluidic system devices, such as microfluidic bio chips, often termed Bio Flips, LOCs and microTASs, to overcome at least one drawback of the prior art mentioned above. Further, there is a need to develop technologies that lead to total integration of peripheral functions onto single microchips, including innovative low power/pressure sources for on-chip fluidic manifolds that allows analyzing samples in small volumes of liquid as well as providing more economical use of reagents and samples.

### SUMMARY OF THE INVENTION

The microfluidic device according to the present invention allows the integration of many functions for molecular diagnostics applications. The microfluidic device according to the present invention may analyze samples in small volumes of liquid, providing more economical use of reagents and samples, and in some cases dramatically speeding up assays.

The microfluidic device for molecular diagnostic applications according to the present invention allows a lateral flow micro fluidic channel system. This allows a vertical integration of sensors and other devices for the treatment, processing and/or analysis of a fluid sample of an assay.

To integrate a large number of functions on the microfluidic device for molecular diagnostic applications according to the present invention it is suggested to integrate all or at least most of these functions on at least one substrate having micro channel structures, which are covered by a membrane for fluid transport, as it is explained below.

According to the present invention a micro fluidic device for analysis of a fluid sample for molecular diagnostics applications is provided, comprising:

a substrate having a surface with at least one micro channel structure thereon;

at least one detecting, controlling and/or processing element;

at least one reception chamber for receiving the fluid sample, wherein the reception chamber is formable between a membrane and the substrate, wherein the reception chamber is fluently connected with at least one micro channel;

at least one membrane, wherein the membrane covers the upper surface of at least one micro channel structure arranged on said substrate leakage proof, whereby movement of said membrane causes a pump action on fluid located in said reception chamber in said micro channel and/or causes a valve action on fluid directed through said micro channel; and

at least one device for actuating the movement of the membrane, comprising pressure and/or vacuum generating means.

The microfluidic device and/or the micro channel structure can be designed such, that a number of same or different fluid sample processing, detecting and/or controlling steps can be carried out separate, simultaneous and/or subsequent thereon.

As used herein, the term “detection means” or “detecting element” refers to any means, structure or configuration, which allows one to interrogate a fluid sample within the sample-processing compartment using analytical detection techniques well known in the art. Thus, a detection means may includes one or more apertures, elongated apertures or grooves which communicate with the sample processing compartment and may allow an external detection apparatus or device to be interfaced with the sample processing compartment to detect a fluid sample, also referred to as analyte, passing through the microfluidic device.

The term “fluid sample” is used to refer to any compound or composition, which can be pumped through the micro channel system. The “fluid sample” is preferably a liquid.

### BRIEF DESCRIPTION OF THE DRAWINGS

FIG. 1 is a sectional side view of a microfluidic device comprising a membrane and plungers for fluid transport at a first position,

FIG. 2 is a sectional side view of the microfluidic device of FIG. 1, wherein fluid is forced into the micro channel system to a second position,

FIG. 3 is a fragmentary sectional top view of the microfluidic device of FIG. 1,

FIG. 4 is a schematic view of a microfluidic device with integrated PCR and detection thereon,

FIG. 5 is a sectional side view of a microfluidic device with sample injection,

FIG. 6 is a sectional side view of a microfluidic device with a reagent storage container,

FIG. 7 is a sectional side view of the microfluidic device of FIG. 6, wherein the reagent is released and forced into the micro channel system,

FIG. 8a is a sectional side view of a microfluidic device with an interdigitated electrode structure for electroporation,

FIG. 8b is a sectional top view of a microfluidic device with an interdigitated electrode structure for electroporation,

FIG. 9a is a sectional side view of a microfluidic device with PCR chamber and integrated temperature sensor and heater element,

FIG. 9b is a fragmentary sectional top view of the microfluidic device of FIG. 9a,

FIG. 10 is a sectional side view of a microfluidic device with a lateral flow-through hybridization array,

FIG. 11 is a sectional side view of a micro fluidic device with an integrated pressure sensor,

FIG. 12a is a sectional side view of a microfluidic device with an integrated biosensor,

FIG. 12b is a sectional top view of a microfluidic device with an integrated biosensor,

FIG. 13 is a sectional side view of a microfluidic device, wherein compressed gas and vacuum is used to actuate the membrane to cause fluid transport.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. Thus, for example, reference to “a fluid” may includes mixtures, reference to “a heat device” includes two or more such devices, reference to “a micro channel” includes more than one such channels, and the like.

It has thus been shown that the present invention has provided a new approach for performing pumping and valving operations by a membrane in micro fabricated fluid systems for applications such as medical diagnostic microchips. By the use of the membrane the microfluidic device, also referred to as cartridge, can be effectively utilized as a pump for fluid transport or as a control valve. The membrane has a variable operational capability. A chip scale integrated sample preparation system can be produced utilizing the invention.

The size of the membrane may be selected so that the membrane completely or partly covers the upper surface of the substrate. It is most preferred that the membrane covers the micro channel system. An up and down movement of said membrane cause a pump action or valve action so that fluid located in said micro channel system is transported or stopped in the micro channel system. An up movement of the membrane causes a suction function and a down movement of the membrane forces a fluid sample flow and/or causes a valve function. In order to apply pressure and/or vacuum to the membrane, the membrane is in contact with pressure and/or vacuum means. Pressure means comprising gas pressure and/or mechanical pressure means such as plungers or there like. The pressure and vacuum means are not in contact with the fluid sample since the membrane has a fluid sealing function. The pressure and/or vacuum means actuate the upper surface of the membrane at specific areas so that defined areas of the membrane can be lifted up and down only. It is preferred that the predominant part of the membrane surface is fixed by means of a support plate, also referred to as fixture. The support plate can comprise at least one recess, hole or conduit so that the membrane can be moved up and down. Furthers, a recess of the support plate having no hole or conduit can function to receive a membrane up-movement caused by fluid sample flow. Vacuum and/or pressure means can be operative connected to at least one recess, hole and/or conduit of the support plate to actuate the pump and/or valve function of the membrane. The membrane area having a valve and/or pump function is arranged adjacent and/or above the micro channel so that fluid sample in said micro channel can be forced through. It can be preferred that the micro channel adjacent and/or below the movable membrane areas have an enlarged structure, i.e. the channel design at this places has a chamber, compartment or lake-like form.

In case of using plungers it is preferred that the lower surface size of the plunger/s corresponds with the shape of the micro channel so that a down movement of the plunger contacting the membrane causes a fluid pressure and/or valve action of the membrane. The plunger can be connected with the upper surface of the membrane, the plunger can be part of the membrane, and/or the plunger fits so in a hole, recess or conduit, that a up and down movement of the plunger actuate the pump and/or valve action of the membrane. If the plunger is part of the membrane, the plunger can be hollow so that a squeezing cause a pump and/or valve action. Thus, the membrane can have a flexible plane shape or a flexible pre shaped design. A membrane with a pre shape design is a membrane that forms at least one compartment or chamber, preferably at least two compartments and chambers.

The compartments and/or chambers of the flexible plane membrane (formed due pump/valve function) and/or of the pre-shaped membrane for receiving fluid sample may have a volume of 0.1 to 100 mm³, preferably 0.5 to 25 mm³ and more preferably 1 to 5 mm³.

Due to the pump and/or valve effect of the membrane at defined areas, i.e. at areas where the membrane is not fixed in its position, fluid sample can be transported through a micro channel system or branched channel system to a desired area. Thus, a fluid sample can be transported to a number of different places to be detected, controlled and/or processed. Therefore, the pump system of the present invention may allow a multiple forward and backward fluid transport.

Further, the integrated membrane with pump and valve functions provides a fast fluid transport, a small pump and valve dead volume as well as a low vertical range of manufacture. The small dead volume is one benefit of the microfluidic device according to the present invention. In the present invention the total volume of all the micro channels can be preferably less than 1 vol.-%, preferably less than 0.5 vol.-% and more preferably less than 0.1 vol.-% of the total fluid volume. However, it is possible to reduce dead volumes further by pumping air trough the micro channel at the end of the pumping cycle.

The membrane as used according to the present invention is preferably liquid tight, so that liquid fluid does not penetrate the membrane during operation. It may be preferred that the membrane is flexible and/or elastic. Suitable membrane materials are polymers, preferably natural or synthetic rubbers.

To obtain a good pump and/or valve effect of the membrane it may be preferred that the membrane has a thickness of 1 μm to 1000 μm, preferably 25 μm to 500 μm and more preferably 50 μm to 200 μm. If the membrane is to thin there is a danger of deterioration of the membrane, which may result in leakage of the fluid sample. However, if the membrane is to thick, there is a danger of malfunction of the pump and/or valve effect of said membrane with respect to fluid transportation. Most preferred is a rubber membrane having a thickness between 50 micron and 200 micron.

According to the present invention, a substrate surface is at least partly covered with a polymeric layer. The micro channel structure can be formed in said polymer layer by general known techniques. For example, micro channels can be formed by use of laser ablation techniques. A laser ablation process can be used, because it avoids problems encountered with micro lithographic isotropic etching techniques which may undercut masking during etching, giving rise to asymmetrical structures having curved side walls and flat bottoms. The use of laser-ablation processes to form microstructures in substrates such as polymers increases simplicity of fabrication, thus lowers manufacturing costs. Further, microfluidic devices according to the present invention in low-cost polymer substrates have the benefit to be disposable.

In general, any substrate which is UV absorbing provides a suitable substrate in which one may laser ablate features. Accordingly, microstructures of selected configurations can be formed by imaging a lithographic mask onto a suitable substrate, such as a polymer or ceramic material, and then laser ablating the substrate with laser light in areas that are unprotected by the lithographic mask. EP-A1 0 708 331 is directed to laser ablation techniques and is incorporated by reference herein. However, micro channel can also be formed by etching and micromachining techniques used to form systems in silicon or silicon dioxide materials.

The term “laser ablation” is used to refer to a machining process using a high-energy photon laser such as an excimer laser to ablate features in a suitable substrate. In general, any suitable substrate which is UV absorbing can be used. The excimer laser can be, for example, of the F₂, ArF, KrCl, KrF, or XeCl type.

The microfluidic device according to the present invention can comprise at least one micro channel. Preferably, the microfluidic device comprises a plurality of micro channels, also referred to as micro channel array, formed on a substrate material.

The micro channel structure formed on the substrate can comprises areas where the fluid sample is treated, such as heated, cooled, controlled, reacted, measured and/or analyzed. Further, the micro channel structure comprises areas of pump and/or valve function.

The micro channel can have the form of a channel. However, at places where the fluid sample is subjected to pump or valve effect or treated, such as heated, cooled, controlled, reacted, measured and/or analyzed, the micro channel may have a wider structure, such as a chamber, compartment or lake-like structure.

The substrate material can be selected from the group comprising glass, ceramic, silicon and/or polymer.

The depths of the micro channels may in the range of 5 micron to 200 micron, preferably of 10 micron to 100 micron, further preferred of 20 micron to 50 micron and more preferred 30 micron.

The width of the micro channels at there top opening may in the range of 0.1 micron to 1000 micron, preferably of 1 micron to 500 micron, further preferred of 5 micron to 250 micron and more preferred 100 micron.

In a preferred method micro channels are formed by pattern wise UV exposure of photosensitive polymer layers. The photopolymer is applied by spin coating. After UV exposure the non-exposed parts are washed away during development. Straight sidewalls are obtained. This method avoids problems encountered with micro lithographic isotropic etching techniques.

Accordingly, under the present invention, microstructures of selected configurations can be formed by imaging a lithographic mask onto a suitable substrate, such as a polymer or ceramic material, and then laser ablating the substrate with laser light in areas that are unprotected by the lithographic mask.

A suitable process of manufacture micro channel structures is disclosed in EP-A1 0 708 331, incorporated by reference.

The micro channel structure connects the fluid sample flow path with areas, where the fluid sample is treated, such as heated, cooled, controlled, reacted, measured and/or analyzed. Areas where the fluid sample can be treated comprising the region of a fluid chamber and/or micro channel. For example the micro channel can be designed such, that fluid sample can be treated at a desired position.

Further, the microfluidic device with at least one micro channel preferably comprises a reagent arranged therein, preferably a solid or gel reagent suitable to react with the fluid sample.

In a preferred embodiment, the reagent is present in a microchannel or in a container which is preferably arranged adjacent to a microchannel or area of treatment.

To receive a reagent, the microfluidic device can comprise a pressure release container, wherein the container can be arranged adjacent to the lower surface of said membrane and below a through going hole of the support plate, wherein the lower end of the through going hole is adjacent arranged to the upper surface of the membrane, so that the release container can be opened by subjecting pressure or vacuum, preferably by means of a plunger, through the hole against the upper surface of the membrane. Such release container preferably comprises at least one liquid reagent.

The plungers can be made of plastic, metal, glass and/or ceramic material.

Detecting, controlling and/or processing elements can be arranged adjacent to the fluid sample chamber and/or adjacent to a micro channel. Heaters, sensors, detectors etc. can be integrated by means of thin film technology.

In general, the microfluidic device can comprise electronic device/s such as thin-film electronic devices. The substrate may include a substrate and a plurality of thin-film layers formed on the substrate. Suitable thin-film electronic devices may include electrodes for applying electric fields, sensors, transducers, optical-based devices, acoustic-based devices such as piezo-based oscillators for applying ultrasonic energy, electric field-based devices, and magnetic field-based devices, among others. Sensors may be temperature sensors such as thermocouples, thermistors such as resistive heating devices, p-n junctions, degenerative band-gap sensors, etc., light sensors for example photodiodes or other optoelectronic devices, pressure sensors for example, piezoelectric elements, fluid flow rate sensors for example, based on sensing pressure or rate of heat loss from a heating element, and electrical sensors, among others.

Preferably, electronic device/s comprise detecting, controlling and/or processing means, also referred herein to as elements. Processing means comprising electronic device/s for temperature control of the fluid, electronic device/s for heating and/or cooling the fluid, electronic device/s configured to sense or modify a property of the fluid. Further, a processing mean, also referred to as processing element, comprises a reagent.

The electronic device/s may be disposed so the electronic devices can participate in sample processing and/or monitoring in the fluid micro channel system or compartment. Accordingly, electronic devices may be disposed more efficiently in relation to microfluidic processing chambers, enabling more flexibility in how samples are manipulated. Furthermore, devices that participate in related aspects of microfluidic processing, such as heaters/coolers and temperature sensors, may be disposed in a more cooperative spatial relationship to modify and sense the temperature of substantially the same fluid volume.

Electronic devices, such as thin-film electronic devices, and method to integrate such devices are disclosed in US-A1 20040151629 and incorporated herein by reference.

Preferably, the detecting, controlling and/or processing elements, comprising an electrode, a sensor, a transducer, a heating element, an optical-based device, such as wave guide, a laser, an acoustic-based device, an electric field-based device and/or a magnetic field-based device. Processing elements comprising for example cell lyses, washing, mixing, amplification by PCR and/or detection.

To cause the fluid sample transport according to the present invention the micro channel structure is covered with a membrane, so that fluid sample can be guided or forced through the micro channel/s. At least one membrane can partly or completely cover the micro channel structure. It is preferred, that the membrane is connected to the micro channel structure leakage free, so that fluid sample cannot accidental be lost.

In more detail, the microfluidic device having an array of micro channels arranged on said substrate, wherein each of said micro channels being liquid tight covered by a membrane, the membrane is mounted by a support plate, the support plate possesses at least one through going hole, preferably at least two through going holes for each micro channel. Preferably at least two micro channels are operatively connected, whereby movement of said membrane area faced to the lower end opening of the through going hole by means of pressure or vacuum, preferably by means of said plunger, causes a pumping action on fluid located in said reception chamber in said micro channel or causes a valve action on fluid directed through said micro channel.

The microfluidic device according to the present invention is preferably a disposable cartridge. However, the microfluidic device can be made of a disposable cartridge covered with a support plate. The support plate may be reusable or disposable.

The microfluidic device or cartridge can have a connector on at least one surface side, which provides electrical contact, for example with a control system.

It can be preferred, that the membrane is mounted to the substrate by means of at least one support plate, wherein the support plate possesses at least one hole and preferably a plurality of through going holes. The through going holes can have the shape for receiving a plunger and/or for applying pressure or vacuum for actuating the membrane. Further, through going holes of the support plate can be used for cooling actions, for detection and/or for controlling purposes.

The microfluidic device according to the present invention can be used as Lab-on-chip (LOC) or as Micro Total Analyses Systems (micro TAS) in for example molecular diagnostics applications.

The microfluidic device according to the present invention comprises according to one embodiment at least two elements: (a) a substrate with thin film micro channel structures, integrated electrical and optical function, such as sensors and actuators, and the electrical infrastructure, and (b) a membrane, which covers leakage tight the micro channel structures.

According to a further embodiment of the invention, the substrate and membrane are leak tight pressed together by a support plate. This support plate, also referred as fixture, is part of the microfluidic device and is provided with a number of holes and plungers that fit into these holes. The plungers initiate fluid transport by actuating the rubber membrane causing a pump and/or valve action, so that liquid is forced into the micro channels on the substrate and forced to the next treatment step of the assay. The fluid actuation system by membrane according to the present invention is fast and provides a small dead volume.

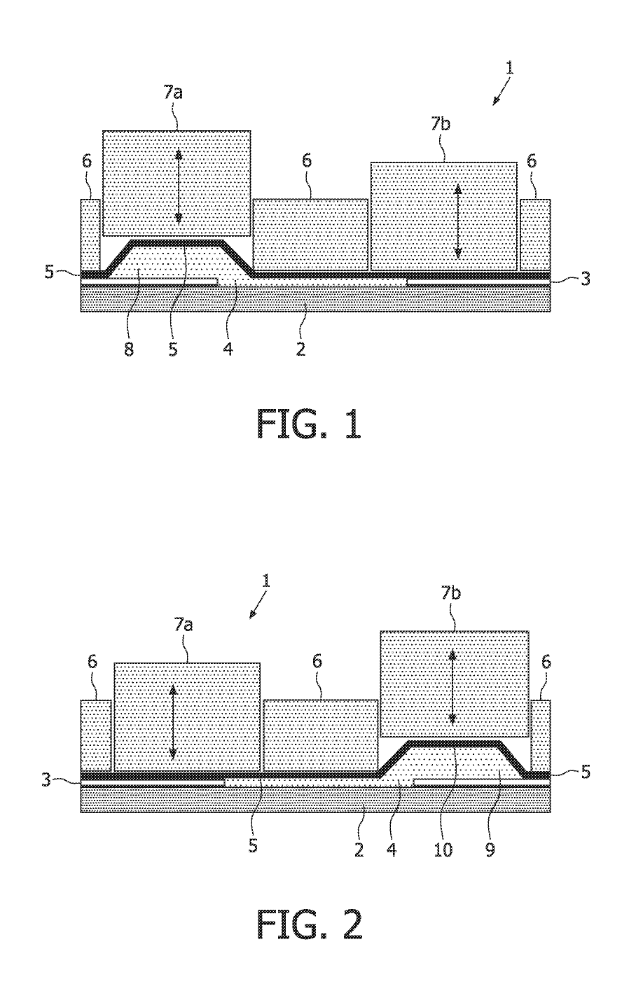

As illustrated in FIG. 1 a microfluidic device (1) according to the present invention comprises a substrate (2) with a polymer layer (3), wherein the micro channel structure/s (4) is formed in said polymer layer (3). The micro channel structure/s (4) are covered with a flexible membrane (5). The membrane (5) is liquid tight connected to the substrate (2) by means of a support plate (6). The support plate (6) comprises through going holes into which plungers (7a/7b) are engage. The plunger (7a) is in an up position for pumping action and plunger (7b) is in a down position for valve function. Below the plunger (7a) and between the flexible membrane (5) and the substrate (2) with polymer layer (3) a fluid sample chamber (8) is formed. Downward movement of the plunger (7a), for example by pressing, forces the fluid sample from the fluid chamber of first position (8) into the micro channel structure (4).

The fluid sample can be forced by actuating the membrane to a desired treatment step. Due to the pump and valve function of said membrane it is possible to force the fluid sample to any desired location of the micro channel structure.

FIG. 2 shows the microfluidic device (1) of FIG. 1, wherein the fluid sample is forced due to the downward movement of the plunger (7a) and the resulting pump action of the membrane (5) into the micro channel structure (4), whereby a fluid sample chamber (9) is formed, so that the fluid sample can be processed. The membrane (5) is liquid tight connected to the substrate with polymer layer (3) having a micro channel structure (4) by means of a support plate (6). As can be seen, the membrane has a valve action at the downward position of the plunger (7a), whereas the membrane at position (10) below the plunger (7b) can cause a pump action if necessary, e.g. if the fluid sample has to be further treated.

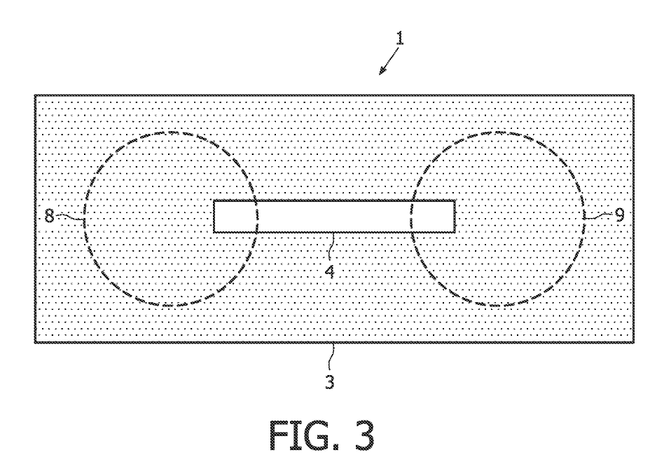

FIG. 3 is a fragmentary sectional top view of the microfluidic device of FIG. 1. The micro channel structure (4) formed in said polymer layer (3) of the substrate (2) can be seen. The micro channel structure (4) connects the fluid chambers (8) and (9). At the bottom of at least one fluid chamber (8) and/or fluid chamber (9) a processing element, a detecting element or treatment element can be arranged.

For example, heaters, temperature sensors and/or detectors can be easily integrated onto the substrate adjacent to the fluid chamber by using thin film technology. However, processing and/or detecting elements can be applied by any suitable technology known in prior art.

The microfluidic device according to the present invention can comprise at least one integrated PCR processing area and at least one detection area connected with said micro channel structure. The fluid sample transport is caused by the pump and valve function of the membrane. The detection area, processing area and/or micro channel structure can have at least one integrated heater and/or temperature sensor. A connector at a side of the microfluidic device or cartridge provides electrical contact with a control system.

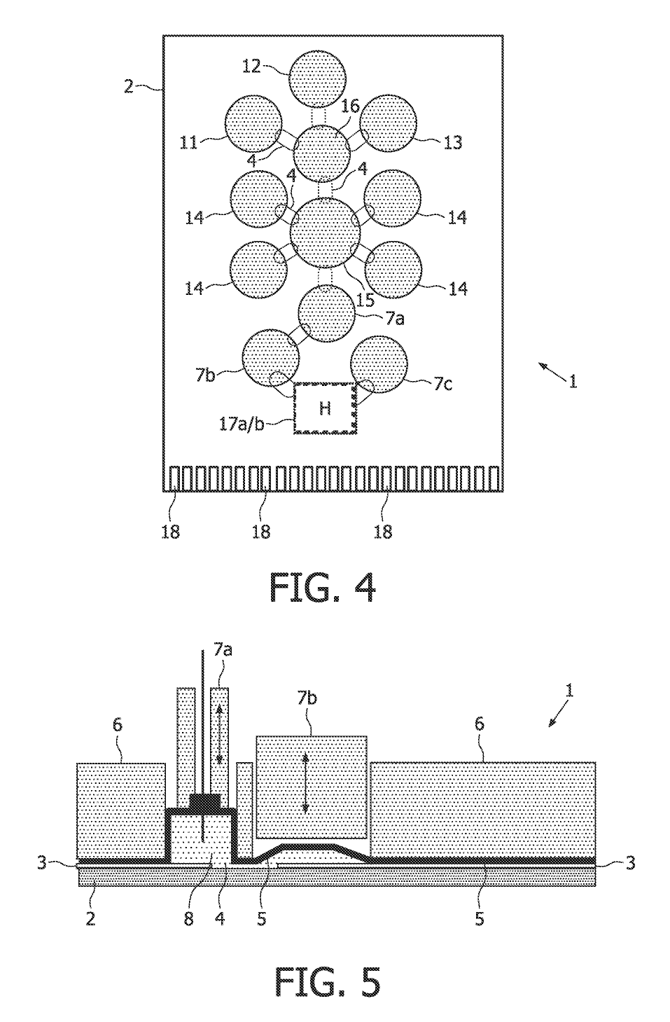

A preferred embodiment of the microfluidic device contains 3 sample injection ports, 4 PCR chambers with integrated heaters and temperature sensors and a lateral flow-through hybridization detection array with integrated heater and temperature sensor (see FIG. 4). The lateral flow-through of the fluid sample allows that detecting elements can be easily arranged below or above the lateral area.

FIG. 4 shows an example of a microfluidic device (1) with combined PCR and detection elements. The substrate (2) and the membrane (not shown), which is preferably a rubber membrane, are leak tight pressed together by a fixture of a support plate (not shown) with through going holes for receiving plungers in order to force the fluid probe by valve und pump action (7a/7b/7c) of said membrane through the micro channel structure (4) to the desired area of treatment. The microfluidic device (1) comprises a sample injection port (11), a master mix injection port (12), a spare injection port (13), four PCR chambers (14), a central distribution chamber (15), membrane (16) with pump and valve function, a lateral flow-through hybridization array (H) with integrated heater (17a) and temperature sensors (17b) and electrical contacts (18). The support plate comprises additional holes for air-cooling of the PCR chambers and a hole for viewing/controlling of the hybridization array (not shown). The other part of the fixture is provided with a number of holes and plungers that fit into these holes. According to this embodiment the fixture is not part of the cartridge but belongs to the readout and control instrument. However, the fixture or support plate can be part of the microfluidic device. Micro channels on the glass plate connect the different chambers. The rubber membrane has pre-shaped cavities for fluid injection and reagent storage. The plunger system can actuate the membrane for fluid pump as well as valve functions. Advantage of this fluid actuation system is fast fluid transport and small dead volumes.

For processing a fluid sample, the fluid sample has to be placed below the membrane, e.g. between the upper surface of the substrate and the lower surface of the membrane. According to a preferred embodiment of the present invention, the fluid sample is placed under the membrane by means of an injection. According to an alternative embodiment of the present invention, the microfluidic device has at least one sample port for receiving a fluid sample. The receiving port can be integrated in the membrane. Preferably, the receiving port can be opened and sealed.

One embodiment for sample injection is shown in FIG. 5, wherein the microfluidic device (1) comprises a substrate (2) with a polymer layer (3), wherein the micro channel structure/s (4) is formed in said polymer layer (3). The micro channel structure/s (4) is covered with a flexible membrane (5). The membrane (5) is liquid tight connected to the substrate (2) by means of a support plate (6). The support plate (6) comprises through going holes into which plungers (7a/7b) are engaged. The plunger (7a) is in an up position for pumping action. The plunger (7a) has a channel for receiving a needle. Plunger (7b) is in a further down position. Below the plunger (7a) and between the flexible membrane (5) and the substrate (2) with polymer layer (3) a fluid sample chamber (8) is formed. As can be seen in FIG. 5 the membrane has a region of increased thickness at the top facing to the channel lower end opening of the plunger (7a). Further, the rubber membrane is cylindrical shaped at this position. A disposable (metal or plastic) hollow plunger is placed on top of the membrane. A needle that is pinned through the thick part of the membrane introduces the sample into the fluid chamber (8). Moving down the plunger (7a) will force the injected fluid sample into the micro-channel system (4).

For processing the fluid sample, it can be suitable to treat or preferably react, the fluid sample with at least one reagent. To provide a ready to use microfluidic device it may be preferred that the microfluidic device according to the present invention comprises at least one container that can release a component, for example a reagent, when opened. The container can be constructed and arranged such that it opens due to heat action and/or pressure action of the membrane. It is preferred, that the container is arranged adjacent to an area of treatment and/or adjacent to the micro channel structure so that the reagent can contact the fluid. The reagent is preferably a solid or liquid component. The liquid component can comprise a gel and the solid component can be a powder or wet powder to facilitate and speed up a reaction with the fluid sample.

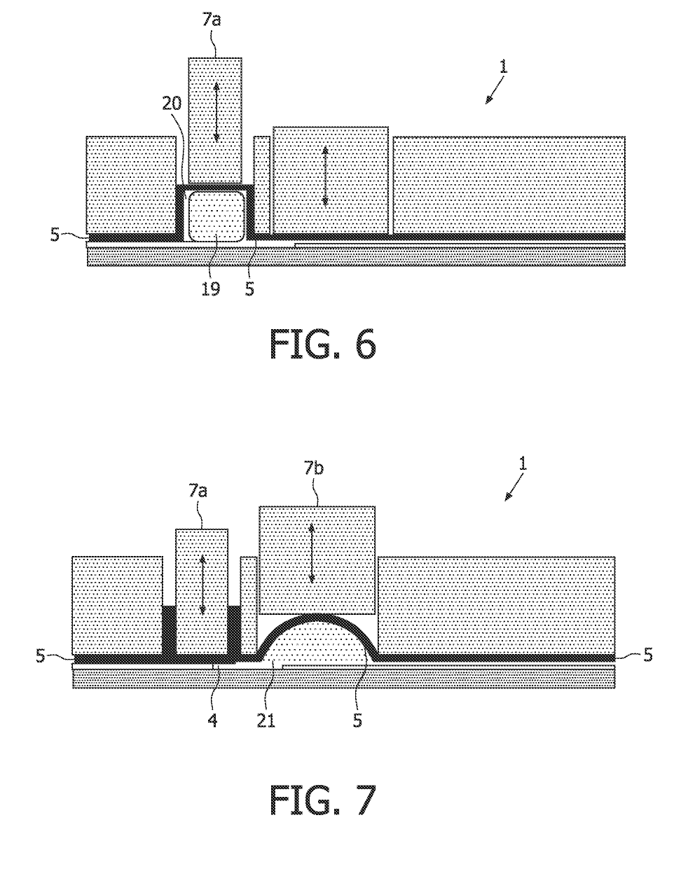

An embodiment of a microfluidic device (1) comprising a container to store for example liquid reagents is shown in FIG. 6.

According to FIG. 6 the reagents are sealed in a thin plastic container (19) and placed in the cylindrical cavity (20) of the membrane (5). The container (19) can be provided with an easy pressure and/or heat openable part. Moving down the plunger (7a) cause a break of the plastic container in a controlled way and the reagent is released and forced into the micro-channel system (4) due to the forcing action of the membrane (5).

FIG. 7 illuminates the fluid transport of the microfluidic device (1) according to FIG. 6 by means of the pump-action of the plunger (7a) to the next fluid chamber (21) where the reacted fluid sample with the released reagent from the container is further treated and/or analyzed. By means of the plunger (7b) and the pump action caused by the membrane (5) located below, the fluid sample can be forced through the micro channel structure (4) to the next place of treatment.

A further preferred embodiment according to the present invention comprises a microfluidic device with an interdigitated electrode structure, which can be used for cell lyses. Cells can be forced through the micro channel structure due to the pump and valve action of the membrane. A treatment area of the micro channel structure can comprise an interdigitated electrode structure. Voltage applied by the electrode structure will cause locally high electrical fields and thereby disrupting the cell membrane and releases the DNA. This method of cell lyses is called electroporation.

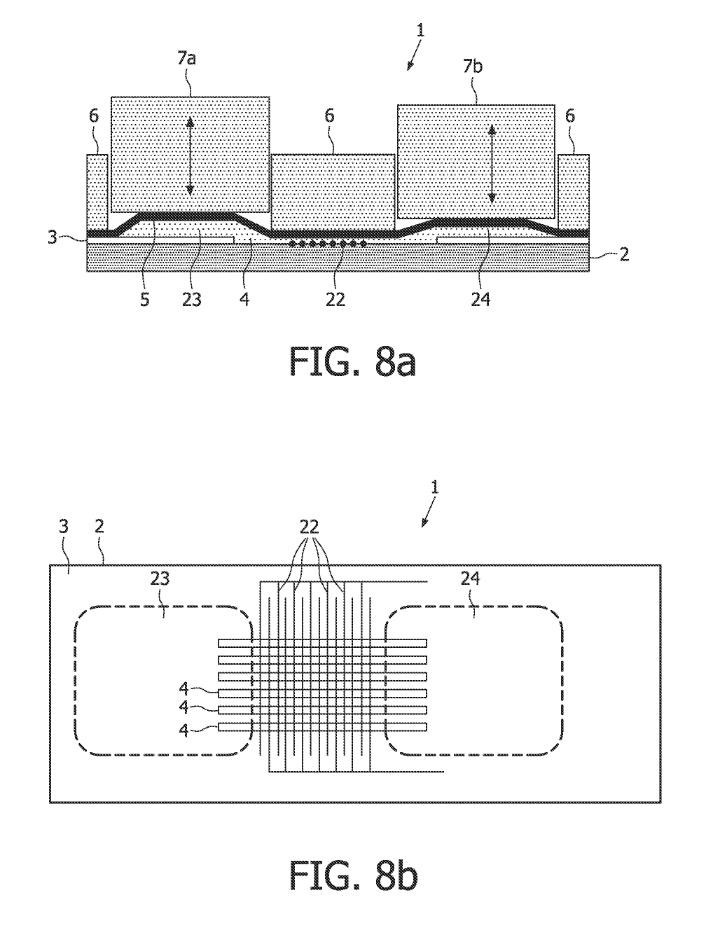

FIG. 8a (side view) and 8b (top view) show a microfluidic device (1) according to the present invention, which can be used for cell lyses, comprising a substrate (2) with a polymer layer (3), wherein the micro channel structure/s (4) is formed in said polymer layer (3). The micro channel structure/s (4) is covered with a flexible membrane (5). The membrane (5) is liquid tight connected to the substrate (2) by means of a support plate (6). The support plate (6) comprises through going holes into which plungers (7a/7b) are engage. The plungers (7a) and (7b) are moved up and down in an alternating way so that the fluid is forced back and forth trough the micro channel structure. The micro channel structure comprises an interdigitated electrode structure (22) placed between two fluid chambers (23) and (24).

Voltage differences over the electrode structure disrupt the cell membrane and releases the DNA, which can be further treated and/or examined.

Providing sharp tips to the interdigitated electrode structure and locally shielding the electrodes with an insulating material, e.g. SiO2, can increase the electrical field strength.

Thus, the microfluidic device of FIG. 8 allows forcing cells by the membrane through the micro-channels. At least one channel can be provided with an interdigitated electrode structure. Such cell lyses process, called electroporation, can easily be integrated in the cartridge, i.e. microfluidic device, of the present invention.

Another embodiment of a microfluidic device according to the present invention used for cell lyses operates by pumping the liquid back and forth by the membrane through a chamber with sharp silica beads. The size of the beads should be selected such that the beads are locked by the grating structures in the micro channels. In general, the beats are of 10 μm to 50 μm and preferably 25 μm.

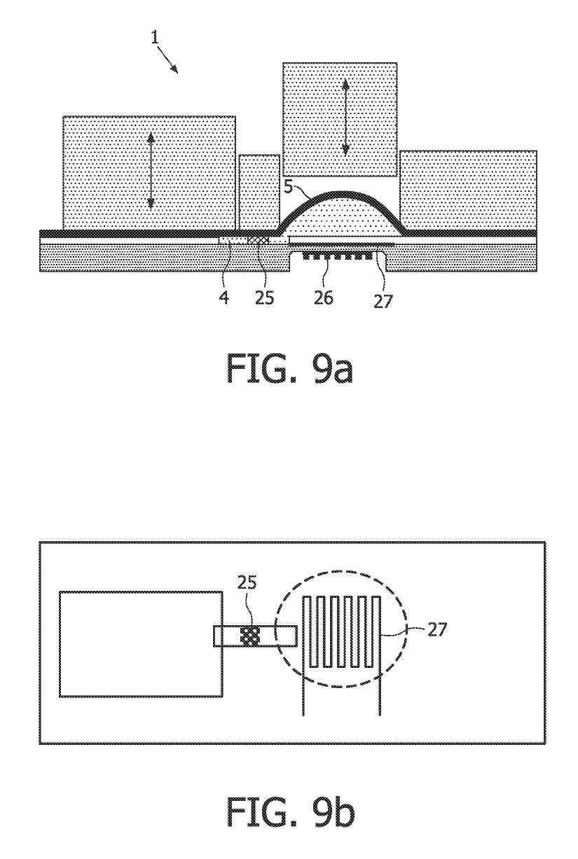

A further preferred embodiment of the present invention is a microfluidic device with at least one PCR treatment area, preferably a PCR chamber, at least one integrated temperature sensor and at least one heater element, all adjacent arranged and/or part of the micro channel structure. A side view and top view of one example of such a microfluidic device is shown in FIG. 9a (side view).

As can be seen from microfluidic device (1) of FIG. 9a (side view) fluid is forced by the membrane into the micro channel (4) and than towards the PCR chamber (21). In the micro-channel (4) a fine micro pattern (25) can be formed. This structure, preferably a porous structure (25), is used to store dry reagents, for example PCR primers, which can be applied by inkjet printing technology or other known technologies. The reagents can be coated on the microstructures and/or can be absorbed. The fluid flow of the fluid sample along said microstructures takes up the reagents and transports it into the PCR chamber (21). Heater and temperature sensor elements (27) are arranged at the bottom of the PCR chamber (21). Further, the substrate (2) comprises a cooling element (26) in form of a recess.

Heater elements and temperature sensor elements can be protected by a thin dielectric layer and/or by a 30-micron thick polymer layer. However, a polymer layer is preferred, since SiO₂ and Si₃N₄ are known as PCR inhibitors.

As mentioned above, the microfluidic device according to the present invention can comprise at least one cooling element. The cooling element can be a recess of the substrate. Preferably, the recess is formed on the backside of the substrate, which is opposite to the membrane surface. For having a cooling action cool air can be directed onto the recess of the substrate. The substrate can be a glass plate, a metal plate or a polymer plate. Also a cooler element may be arranged at the microfluidic device of the present invention to enhance thermal contact during cooling. An air-cooling element can be placed at any place where cooling action is desired, preferably a cooling action element is placed adjacent or at the PCR chamber.

Another alternative to provide heating and cooling can be obtained by use of Peltier elements. The Peltier elements can be attached to the backside of the substrate.

Further, to increase temperature rise speed, such as ramping speed, it is necessary to reduce the thermal mass at the location of the PCR chamber. This can be achieved by local thinning of the substrate, for example the glass plate, by e.g. wet etching or powder blasting.

FIG. 9b is a fragmentary sectional top view of the microfluidic device (1) of FIG. 9a, having a PCR chamber (21) with integrated temperature sensor and heater element (27). The PCR chamber (21) is connected with the micro channel structure (4). In front of the PCR chamber (21) a dry reagent/s absorbed onto a porous micro structure (25) is placed. At the PCR chamber a heater (27) and temperature sensor element (27) are arranged schematically indicated by the meandering resistor wire (27). The fluid sample is mixed with the reagent/s when the fluid sample reaches the area where the dry reagent/s are placed. The fluid sample flow along the micro channel structure is forced by the membrane pump and valve action as already described before. Further, the microfluidic device according to the present invention can comprise at least one cooling element. The cooling (26) element can be formed as or in a recess of the substrate.

A further preferred embodiment of the present invention comprises at least one detection element, preferably an optical detection element. The detection element can be arranged adjacent and/or at the micro channel structure. It is preferred that the detection element is arranged at the last step of the assay of the microfluidic device of the present invention, since it is common that the last step in the assay is detection.

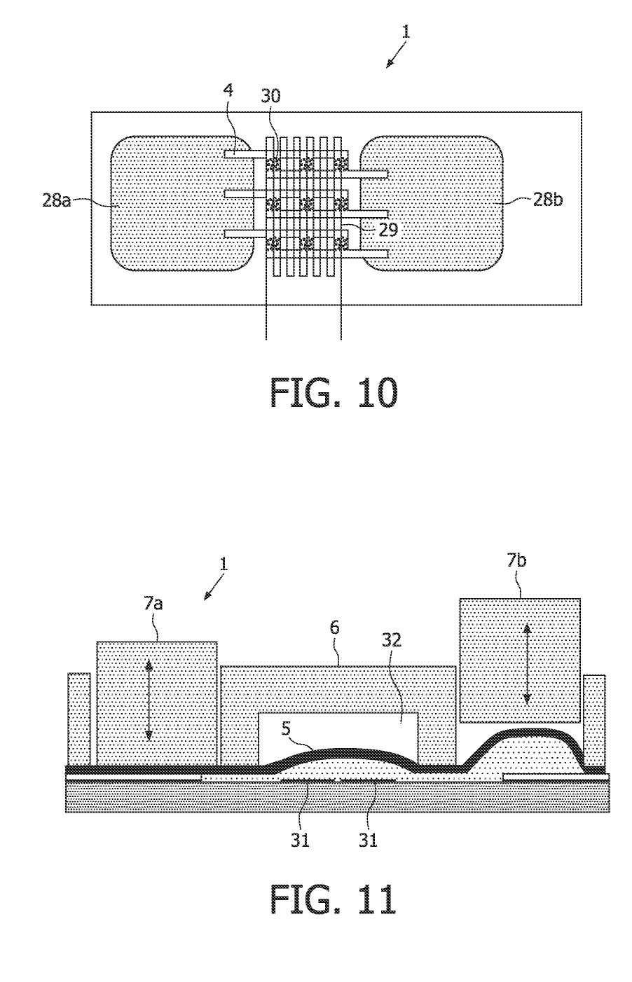

FIG. 10 shows a lateral flow-through hybridization array of a microfluidic device (1) according to the present invention. As can be seen the fluid to be analyzed is forced into the micro-channel structure (4) due to pump and valve action of the membrane (not shown) at places (28a/28b) along the processing area with integrated heater (29), whereby the probe area comprises fine micro-structures (30).

The probe area/s can be provided with fine micro-structures, which can be obtained by using standard lithography. The micro-structures are used to place and/or fix the reagent/s, in this case the hybridization probes, which are applied e.g. by inkjet technology. It is also possible to locally obtain extreme sub-micron structures, for example in the range of 100 nm, by applying an additional exposure step using laser beam interference technology. Another alternative embodiment of the microfluidic device can comprise individual chambers containing small porous substrates.

Read out of the hybridization array can be obtained by florescent detection using laser illumination and a CCD camera arranged at the rear side of the substrate opposite to the membrane. For a camera observation and/or detection it is preferred that the substrate is transparent.

An alternative is to use photo activated polyacrylamide gel. Porous plugs of this material are formed by local UV exposure and unpolymerized material can be removed by washing.

According to the microfluidic device of the present invention it is possible to integrate a number of various functions. Functions that can be integrated comprising mixing, magnetic bead transport, cell manipulation, cell counting and/or capillary electrophoresis as detection method.

The microfluidic device of the present invention can also comprise a camera. It is preferred to arrange the camera at the rear panel of the substrate opposite to the membrane. For a camera observation and/or detection it is preferred that the substrate is transparent. Further, the thickness of the substrate at the place where the camera is arranged can be reduced to improve the optical recording of said camera. A microfluidic device with a camera can be used for example in combination with a cell manipulator function and/or cell counting function.

Further, a channel leakage detector, air in channel detector, mass flow sensor and/or pressure sensor can be integrated on the microfluidic device of present invention.

A channel leakage detector can comprise unshielded electrodes which can be integrated at the substrate for example in a groove adjacent with the fluid flow micro channel system. A resistance change can be used to indicate a leakage.

Air in the micro channel system of the microfluidic device according to the present invention can be detected for example by a change of the capacitance of a SiO₂ shielded interdigitated electrode structure. It is preferred to integrate the SiO₂ shielded interdigitated electrode structure on the substrate of the microfluidic device in contact with the fluid flow micro channel system. Sensitivity can be improved by differential measurement with an equal capacitive structure that is placed adjacent to the micro channel.

A mass flow sensor can be based on a differential measurement of resistance of a heated wire. The fluid sample flow takes up heat so that the temperature change results in a resistance change with can be measured. It is preferred to integrate the mass flow sensor on the substrate of the microfluidic device adjacent or in contact with the fluid flow micro channel system.

FIG. 11 shows a microfluidic device (1) with a capacitive pressure sensor (31) arranged in the micro channel on the substrates at a region below the membrane (5). The support plate (6), also referred to as fixing element, has a recess (32) so that the membrane can move up and down by actuating the plungers (7a/7b) corresponding to the actual pressure. The capacitive sensor senses the amount of liquid above the sensor surface, which is a measure for the actual pressure.

Besides electrical sensors and detectors, optical detection elements can also easily be integrated. For example light can be coupled in an out by total internal reflection (TIR) on an inclined surface at the beginning or ending of a wave-guide, which is part of the microfluidic device according to the present invention. The inclined surface can be obtained by an additional lithographic exposure step with an inclined exposure beam as already known in prior art or by using photo-masks with incorporated phase gratings, also known in prior art. It is also possible to integrate the photo detector on the substrate of the microfluidic device by using additional photo mask steps and processing “Low Temperature Poly-Silicon” LTPS, but this may make manufacture of the substrate more complex and expensive. Integrated wave-guides can be suitable used to control and/or analyze processed fluid sample of the hybridization array.

The microfluidic device according to the present invention further allows the integration of external components like Si biosensors. The membrane can be perforated to provide an electrical contact of the Si device with the interconnection circuitry on the substrate, preferably a glass plate. The silicon device is provided with Au bumps and the rubber membrane acts as a seal ring to the electrical contacts at the perforated points. The Si device can be attached to the glass substrate by ultrasonic bonding, thermo compression bonding, and/or laser welding.

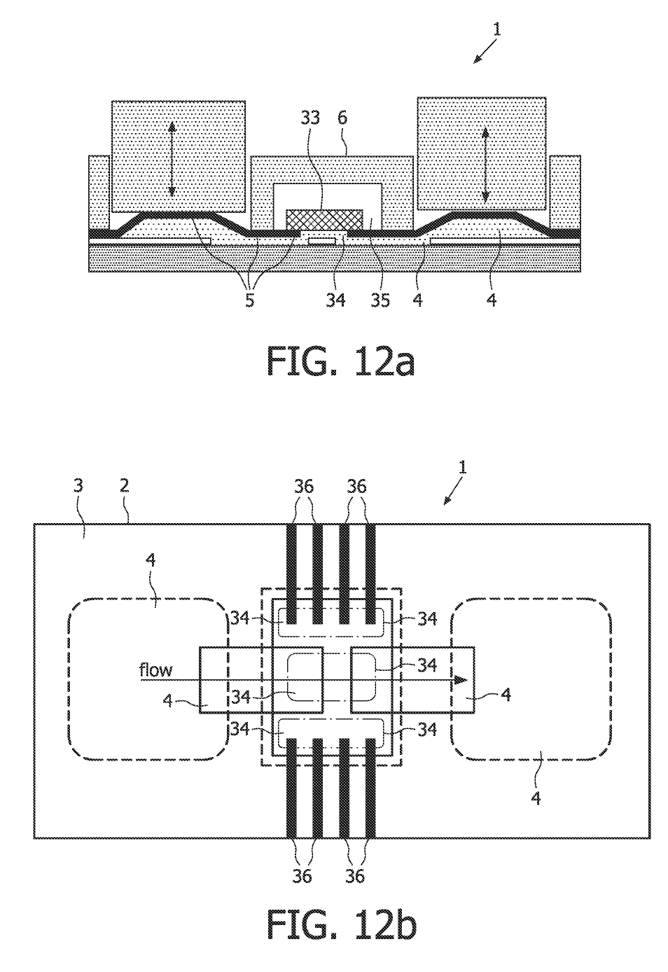

FIG. 12a (side view) and 12b (top view) show a microfluidic device (1) with an external device such as a silicon device (33) arranged to cover the top surface of a pore or holes (34) of the membrane (5). The support plate (6), also referred to as fixing element, has a recess (35) for the reception of the silicon device (33). Fluid sample can be forced by the pump and valve action of the membrane (5) along the micro fluidic channel (4) to be detected by the silicon device (33).

Further, FIG. 12b shows interconnecting lines (36) arranged on the substrate (2) covered with a polymer layer (3).

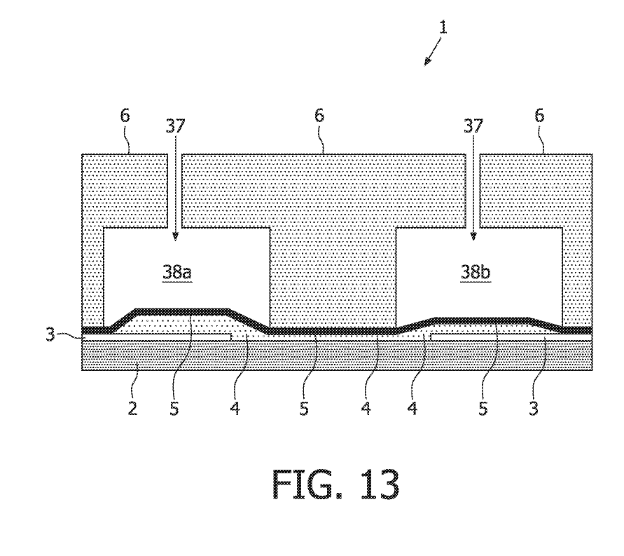

The pump and valve action of the membrane can be actuated by plunger actuation as already described before. However, as an alternative to fluid transport by plunger actuation it is possible to apply fluid pressure, such as compressed air and/or vacuum in order to actuate the membrane of the microfluidic device according to the present invention. An example of a microfluidic device according to the present invention with a membrane actuated by gas pressure instead of plunger actuation is schematically shown in FIG. 13.

FIG. 13 shows a microfluidic device (1) according to the present invention comprises a substrate (2) with a polymer layer (3), wherein the micro channel structure/s (4) is formed in said polymer layer (3). The micro channel structure/s (4) is covered with a flexible membrane (5). The membrane (5) is liquid tight connected to the substrate (2) by means of a support plate (6). The support plate (6) comprises through going holes (37) to which pressure means and/or vacuum means can be connected. The support plate (6) further comprises a recess (38a) and (38b) to receive the membrane in an up position, where the membrane (5) forms a fluid sample chamber. The recess (38a) and (38b) is operatively connected with the through going holes (37). The valve and pump action of the membrane (5) can be actuated at the recess areas (38a) and (38b) separate from each other by actuation of the pressure means and/or vacuum means (not shown) connected to the through going holes (37) to force the fluid sample through the micro fluid channel system (4).

The microfluidic device according to the present invention can be used for fluidic/electronic/mechanical devices in biomedical applications such as microTAS and LOC, biosensors, molecular diagnostics, food and environmental sensors. Further it can be used for the synthesis of chemical or biological compounds.

Preferably, the microfluidic device according to the present invention can be used for:

chemical, diagnostic, medical and/or biological analysis, comprising assays of biological fluids such as egg yolk, blood, serum and/or plasma;

environmental analysis, comprising analysis of water, dissolved soil extracts and dissolved plant extracts;

reaction solutions, dispersions and/or formulations analysis, comprising analysis in chemical production, in particular dye solutions or reaction solutions;

quality safeguarding analysis; and/or

synthesis of chemical or biological compounds.

Manufacturing of the glass substrate with micro-channels and integrated functions can be provided by a four mask thin film process as known in prior art.

Examples for the manufacture of the glass substrate with micro-channels and integrated functions are given below:

| | |
|---|---|
| Substrate: | 0.4 mm Schott AF45 |
| Thin film processing | |
| four mask level | |
| Resistor layer: | 100 nm Pt, or Ti, Cr, Ni, Pt, Au, W |
| Conductor layer: | 1 micron Al or Cu, Au, Ag |
| Dielectric layer: | 0.5 micron SiO₂ or SiN |
| Polymer layer: | 30 micron SU8 or BCB, or other |
| | photopolymers |

Resistor elements for heater and temperature sensor are preferably made of the same thin layer such as a Pt. For the temperature-sensing element it may be important that the temperature coefficient of resistance (TCR) of the selected metal is sufficiently high.

Preferably a conductor layer of 1 micron of aluminium is used.

The combination of metals should be selected so to be compatible with the thin film dielectric layer of SiN or SiO2.

Micro channels and structures are made on the substrate, preferably glass or plastic, by standard photolithographic processing using photopolymers such as SU8, supplied by MicroResist Technology, and/or BCB photopolymer supplied by Dow Chemical.

Active electrical functions, such as diodes, transistors, used to control actuators and sensors can be integrated using Low Temperature Poly-Silicon (LTPS) active matrix LCD technology, as known in prior art.

To provide a comprehensive disclosure without unduly lengthening the specification, the applicant hereby incorporates by reference each of the patents and patent applications referenced above.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. A microfluidic device (1) for analysis of a fluid sample, especially for molecular diagnostics applications, comprising:
a substrate (2) having a surface with at least one micro channel structure (4) thereon;
at least one detecting, controlling and/or processing element;
at least one reception chamber (8) for receiving the fluid sample, wherein the reception chamber (8) is formable between a membrane and the substrate (2), wherein the reception chamber (8) is fluently connected with at least one micro channel (4);
at least one membrane (5), wherein the membrane (5) covers the upper surface of at least one micro channel structure (4) arranged on said substrate (2) leakage proof, whereby movement of said membrane (5) causes a pump action on fluid located in said reception chamber (8) in said micro channel (4) and/or causes a valve action on fluid directed through said micro channel (4); and
at least one device (7a/7b/36) for actuating the movement of the membrane (5), comprising pressure and/or vacuum generating means.

2. The microfluidic device (1) of claim 1, wherein the micro channel structure (4) is formed in a polymer layer (3), glass or ceramic layer (3), wherein said micro channel structured polymer layer (3), glass or ceramic layer (3) is arranged on the substrate (2) surface.

3. The microfluidic device (1) of claim 1, wherein the membrane (5) is mounted to the substrate (2) by means of at least one support plate (6), wherein the support plate (6) possesses at least one hole, preferably a plurality of through going holes, suitable for receiving a plunger (7a/7b) and/or suitable for applying pressure or vacuum for actuating the membrane (5).

4. The microfluidic device (1) according to claim 1, wherein the device comprises a reagent arranged therein.

5. The microfluidic device according to claim 4 wherein the reagent is a solid or gel reagent suitable to react with the fluid sample.

6. The microfluidic device (1) according to claim 4 wherein at least one micro channel (4) contains a reagent.

7. The microfluidic device (1) according to claim 4, wherein between at least one membrane (5) and at least one micro channel structure (4) is at least one heat and/or pressure release container (19), whereby the container (19) is adjacently arranged to an area of treatment and/or to a micro channel (4), and wherein said container (19) comprises at least one reagent.

8. The microfluidic device (1) according to claim 7 wherein the container comprises at least one liquid reagent.

9. The microfluidic device (1) according to claim 7, wherein the pressure release container is arranged adjacent to the lower surface of said membrane (5) and below a through going hole of the support plate (6), wherein the lower end of the through going hole is adjacent arranged to the upper surface of the membrane (5), so that the release container (19) can be opened by subjecting pressure or vacuum, preferably by means of a plunger (7a), through the hole against the upper surface of the membrane (5).

10. The microfluidic device (1) according to claim 1, having an array of micro channels (4) arranged on said substrate (2), each of said micro channel (4) being liquid tight covered by a membrane (5), wherein the membrane (5) is mounted by a support plate (6), the support plate (6) possesses at least one through going hole, preferably at least two through going holes for each micro channel (4) and preferably at least two micro channels (4) are operatively connected, whereby movement of said membrane (5) area faced to the lower end opening of the through going hole by means of pressure or vacuum, preferably by means of said plunger, causes a pump action on fluid located in said reception chamber (8) in said micro channel (4) or causes a valve action on fluid directed through said micro channel (4).

11. Use of the microfluidic device (1) according to claim 1 for:
chemical, diagnostic, medical and/or biological analysis, comprising assays of biological fluids such as egg yolk, blood, serum and/or plasma;
environmental analysis, comprising analysis of water, dissolved soil extracts and dissolved plant extracts;
reaction solutions, dispersions and/or formulation analysis, comprising analysis in chemical production, in particular dye solutions or reaction solutions; and/or
quality safeguarding analysis.
